(19) United States
(12) Patent Application Publication
(10) Pub. No.: US 2003/0216688 A1
(43) Pub. Date: Nov. 20, 2003

(54) Cooling cannula system and method for use in cardiac surgery

(76) Inventors: Huybregts M.A.J.M., Ouderkerk a/d Amstel (NL); Robert J. Todd, Salt Lake City, UT (US); Alison S. Curtis, Riverton, UT (US)

Correspondence Address: Nicole S. Bradley, Edwards Lifesciences LLC, Law Dept., One Edwards Way, Irvine, CA, 92614, US

(21) Appl. No.: 10/151,354

(22) Filed: May 20, 2002

(51) Int. Cl. A61F 7/12

(52) U.S. Cl. 604/113; 604/96.01; 604/500; 428/35.2; 264/454

(57) ABSTRACT

A novel improved enhanced cardiac surgical method yields unexpected results by having an enhanced intraluminally emplaced cooling system. In preferred device embodiments improvements include a first means for draining venous blood from at least one of the right atrium, superior vena cava and inferior vena cava and an improved means for cooling involved luminal surfaces. Tissue insult and injury is substantially mitigated by engagement of the cooling means with select aspects of involved atrial tissue to facilitate transfer of heat. Methods for making embodiments of the invention are provided. A method for the treatment of cardiac disease using the improved enhanced cardiac surgical cooling system, and an article of manufacture, comprising packaging material and the device are also taught. Likewise, the alternate cooling mechanisms disclosed have applicability both on- and off-pump in a variety of procedures ranging from traditional open cardiac surgical repair and by-pass to endovascular procedures using percutaneous access and minimally invasive therapies.

### BACKGROUND ART

This invention pertains generally to medical devices and their methods of use for treatment of cardiovascular disease, and more particularly to specialized cannulae useful for implantation and/or emplacement within cavities or blood vessels of the body. The present invention is particularly suited to surgical methods whereby the cannulae facilitate goals of improved surgical methods, for example whereby within the context of known or developed procedures such devices mitigate, extenuate or otherwise positively impact cellular or tissue based insult by having provisions for heat transfer, within any cardio thoracic, cardiovascular, or related procedure.

Current technology addresses needs within the context of the world's most prevalent set of disease states, namely those loosely grouped under the above-referenced moniker, however, it is respectfully proposed that aspects of the instant teachings bridge the gaps between current therapies targeting at least one of heart valve disease (HVD) coronary artery disease (CAD) and congestive heart failure (CHF) in such a way that artisans will readily ascertain and understand the cross-functional utility of the instant teachings. Likewise, it is further submitted that novel approaches set forth herein are expressly de-limited to provide improvements with any appropriate groups of procedures within known or developed therapies used as stand alone or adjunct to conventional cardiac, thoracic and vascular surgery.

The human heart is normally slightly larger than a clenched fist. It is cone-shaped in appearance, with the broad base directed upward and to the right and the apex pointing downward and to the left. The human heart is located in the chest (thoracic) cavity. It is situated behind the breastbone (sternum); in front of the windpipe (trachea), esophagus, and descending aorta; between the lungs; and above the diaphragm. The heart is divided by partitions (septa) into right and left halves, and each half is divided by septa into two chambers. The upper chambers are the atria, and the lower chambers are the ventricles.

The right atrium is a thin-walled chamber receiving blood from all tissues except the lungs. Three veins empty into the right atrium: the first two are the superior vena cava (SVC) and the inferior vena cava (IVC), which bring blood from the upper and lower parts of the body, respectively. The third, the coronary sinus, drains blood from the heart itself. Blood flows from the right atrium to the right ventricle, which in turn pumps blood into the pulmonary artery and ultimately to the lungs.

The left atrium receives the four pulmonary veins, which bring oxygenated blood from the lungs. Blood flows from the left atrium into the left ventricle. Blood is then pumped from the left ventricle through the aorta to all parts of the body except the lungs.

To prevent backflow of blood, the heart is equipped with valves that permit the blood to flow in only one direction. The atrioventricular valves (tricuspid and mitral) are thin, leaflike structures located between the atria and ventricles. The right atrioventricular opening is guarded by the tricuspid valve, whereas the mitral valve guards the left atrioventricular opening. The semilunar valves are pocketlike structures attached at the point at which the pulmonary artery and the aorta leave the right and left ventricles, respectively. While known treatment modes and modalities are effective at addressing valvular surgical needs such as repair and replacement of defective failed or failing valves, vast room for improvement exists within the macro-context of the surgical procedures themselves, in terms of the impact on crucial cellular, tissue-based and organ system level insults.

The heart possesses a vascular system of its own, called the coronary arterial system. It comprises two major coronary arteries—the right and left. These arteries originate from the right and left aortic sinuses (the sinuses of Valsalva), which are bulges at the origin of the ascending aorta immediately beyond the aortic valve. Venous blood from the heart is carried through veins to the coronary sinus, which empties into the right atrium between the IVC and the AV orifice.

The pacemaker of the heart is the sinoatrial node (SA node). This highly important structure is a small strip of specialized muscle located in the posterior (back) wall of the right atrium, immediately beneath the point of entry of the SVC. After an action impulse is generated by the SA node, the impulse immediately spreads through the atrium and is relayed to the atrioventricular node (AV node), located in the lower part of the right rear atrial wall. The coordinated functioning of the SA node and the AV node are responsible for the regulated contractions of the normal heart.

A leading source of morbidity and mortality in Western-style societies is the above referenced general category of cardiovascular disease. Significantly, the categories of each of HVD, CHF, and CAD remain prominent and ostensively seem to be manifested in ever growing segments of the patient populace. It is becoming apparent that there is often a strong correlation between these three categories and peripheral vascular disease (PVD), and those showing signs of at least one likely have symptoms of the other three.

CAD can be manifested in a number of ways. The risks and discomfort associated with angina and ischemia can be produced by the impaired blood flow resulting from CAD. In HVD, CHF, and CAD clinicians are seeing instances of major adverse cardiac events (MACE) such as, but not limited to myocardial infarction resulting from acute blockade of coronary blood flow, producing damage to myocardial tissue, death, stroke and other lifestyle destructing results and eventualities.

Treatment of HVD, CHF, and CAD have been accomplished through a plurality of different approaches. Pharmacological treatment of early symptoms with medicines or diet and lifestyle modification is a threshold first step designed to ameliorate the underlying disease process. It is becoming clear that this most be considered in complement with both radical surgical techniques and endovascular treatment of the coronary blockage, which may be accomplished through the use of devices for balloon angioplasty, atherectomy, laser ablation, stenting, and the like.

Most of the time where pharmacological intervention or endovascular treatment have not fully addressed the involved issues, or are likely to proceed less completely as treatment regimens then hoped, coronary artery bypass grafting (CABG) procedures become necessary. Worldwide, more than 500,000 patients suffering from disabling heart disease are annually afforded the benefits of therapeutic CABG surgery.

A common operation is one in which lengths of superficial veins are taken from the legs and inserted between the aorta and a part of a coronary artery below the obstructive atheromatous lesion. Multiple grafts are often used for multiple atheromatous occlusions. Such multiple grafts are referred to as “triple bypass” or “quadruple bypass” operations, for example. The internal mammary arteries are also used to provide a new blood supply beyond the point of arterial obstruction; however, since there are only two internal mammary arteries, their use is limited.

In undertaking CABG surgery, cardiopulmonary bypass (CPB) is often used as it has proven in many cases to be the most efficient way to achieve the involved surgical goals. Here, the goals are to provide life support functions for the patient, and to provide a motionless, decompressed heart, as well as a dry, bloodless surgical field for the surgeon. CPB may be accomplished by use of large drainage tubes (cannulae and catheters) inserted in the superior and inferior venae cavae (the large veins that return the blood from the systemic circulation to the right upper chamber (right atrium) of the heart. CPB may be done by the establishment of a heart-lung life-support system that provides a diversion of oxygen-poor blood from the venous circulation and its transport to a heart-lung machine (generally known as an “on-pump”). There, re-oxygenation and carbon dioxide elimination are accomplished. Additionally, heat transfer—either warming or cooling—of the diverted bloodstream is provided.

The on-pump procedure works whereby processed blood streams are then selectively pumped back to the body and returned to the arterial tree through cannulae introduced in a major systemic artery, such as the femoral artery. Meanwhile, the heart may be opened and the corrective operation performed. This procedure permits a surgeon to operate on the heart for many hours, if necessary.

It is further noted that on the continuum of surgical procedures and involved on pump and “off pump” procedures many different approaches have come to be important or had clinical significance, or likely shall. To these ends, the instant teachings are understood to impact both sub-generic types of procedures, and those having a modicum of skill will readily understand this.

In terms of structure being driven by function, it has become known that the details of the design and construction of cardiac catheters for these procedures is obviously of great importance to their success. Such catheters can, for example, be inserted via the right atrium or via a peripheral vein such as the jugular vein. But the direct insertion of catheters into the right atrium or vena cava can result in direct surgical trauma from the holes cut into these structures for catheter entry.

Such trauma can lead to bleeding, cardiac arrhythmias, air embolism and surgical adhesions. Moreover, the approach requires major invasive breastbone splitting (sternotomy) or rib spreading (thoracotomy). This is undesirable in its own right, but in addition, makes it much more difficult to perform later surgeries in the event that a repeat open-heart surgery (“redo” operation) is required. In patients whose chest has been previously entered via sternotomy or thoracotomy, extensive adhesions are usually present that increase the risk of injury and hemorrhage in subsequent procedures. For these reasons, devices, such as the present invention, that are adapted to peripheral insertion provide significant advantages.

As is known, both on- and off-pump procedures generally, and, CABG and operations on the cardiac valves are complex, delicate surgeries. It is highly advantageous, therefore, to convert the heart to a resting state in which it is flaccid (non-distended) and non-beating. In addition, a dry, bloodless surgical field is highly desirable for the manipulative procedures entailed by cardiac surgery. Moreover, cellular functions are better preserved in the presence of decreased cardiac energy requirements. In order to convert the heart to such a resting state during CPB, a heart paralyzing (cardioplegia) solution is delivered to the coronary circulation to stop the heart.

This delivery can be accomplished by antegrade and/or retrograde methods. In antegrade delivery, the cardioplegia solution is introduced at the aortic root (arterial end of the coronary circulation) and passes into the capillaries of the myocardium. In retrograde delivery, the cardioplegia solution is introduced into the venous circulation at the coronary sinus and passes backward into the capillaries of the myocardium. In about 75% of CPB procedures, antegrade and retrograde delivery procedures are carried out concurrently.

Although cardioplegia is a crucially important concomitant of CPB procedures, its employment entails significant problems. One such problem is the dilution of the blood, or hemodilution, resulting from the introduction of the cardioplegia solution into the circulation. Normally, the amount of cardioplegia solution is so regulated as to result in a hemodilution amounting to approximately 30-40% cardioplegia solution and 60-70% blood. In general, cardioplegia solutions comprise an aqueous solution of electrolytes and nutrients; normally a relatively high concentration of potassium electrolytes is required to stop the heart. This substantial hemodilution by a non-physiological solution has the obvious potential for tissue damage. For example, adverse mental effects, such as the memory impairment exhibited by some patients who have undergone CPB, may be an adverse effect of cardioplegia solutions.

Organs of the human body, such as the brain, kidney, and heart, are maintained at a constant temperature of approximately 37° C. Cooling of organs below approximately 35° C. is known to provide cellular protection from anoxic damage caused by a disruption of blood supply or by trauma. Cooling can also reduce swelling associated with these injuries. Cooling of organs can generally be accomplished through whole body cooling to create a condition of total body hypothermia in the range of approximately 20° to 30° C. Whole body cooling can be accomplished, for example, by immersing the patient in ice water. Such total body hypothermia has a number of drawbacks that appear to be caused reflexively in response to the reduction in core body temperature. For example, increased systemic vascular resistance, which can result in organ damage, is one such drawback, and immersing a patient in ice water clearly has its associated problems.

A further difficulty is that the extreme drainage of the body's venous blood system and body cooling to make a heart operation possible can result in putting the patient into a clinical state of shock. Recovery from this state of shock then requires appropriately large transfusions because of the relatively intensified blood flow through organ systems and deregulation of fluid distribution in body compartments. Clearly, whole body cooling would advantageously be replaced by a better method.

An alternative to whole body cooling for protecting the heart is to cool the cardioplegia solution in order to cool the heart itself below normal body temperature. Selective cardiac hypothermia produced by perfusing the heart with a cooled cardioplegia solution is susceptible to temperature dilution by the warm blood, which reduces the effectiveness of this method. Moreover, retrograde delivery is only modestly effective in cooling the right side of the heart, and antegrade delivery is relatively ineffective in patients whose coronary vessels have blockages.

In addition, the coronary circulation and the heart chambers cannot be fully protected from the heat of the circulatory system of the body and the heart surface is in contact with the warmer body of the patient. In the right atrium, even though the total bypass hinders entry of venous blood, thermal insulation is even less favorable, partly due to the thermal radiation from the venous cannula or cannulas that run through it. All of these factors result in a right atrium temperature that is only slightly below the body temperature of the patient. Yet the right atrium, which includes the sinoatrial node and atrioventricular node, should be especially well protected by cooling.

Until recently, the only practical solution for this problem—and it was only modestly successful—was to cool the circulatory blood and thus the entire patient. Lowering the body temperature in order to facilitate heart cooling is, however, disadvantageous for reasons already mentioned. A solution to some of these problems was provided by M. A. J. M. Huybregts in U.S. Pat. No. 5,562,606 by providing cooling means around a cannula adapted to be inserted into the right atrium and associated venae cavae.

The cooling means, however, were limited to aspects of an inflatable balloon adapted to lie against the inside wall of the right atrium. Alternate cooling means, ranging from chemical to electrical to biochemical to evaporative may exist providing an unexpected benefit to these disclosures. The Huybregts' 6-6 patent is incorporated entirely and expressly by reference herein, and was a tremendous step forward in addressing the problems targeted. However, it is respectfully proposed that the instant teachings bring that solution to another level, with unexpected results.

Likewise clinically in surgical practice, several limitations were found in the use of such balloons as the only cooling method. First, the balloon was found to interfere with the manipulation of the heart required for surgery on the backside coronaries. In order to access the backside of the heart for surgical procedures, it is necessary to “flip”, or rotate the heart so that the backside is exposed. In practice, the axis for this rotation is the cannula itself.

However, in spite of stabilizer technology and improved cannulae, using cannulae of the prior art, it was found that there was a substantial risk of damaging structures within the heart during this maneuver. Further, prior art devices were found to be deficient in producing a leak-free isolation of the venae cavae. Still further, prior art devices have been found to produce substantial risk of blockage of the coronary sinus. Even further, prior art devices have been found to produce substantial risk of infringing and hence damaging the tricuspid valve. Even yet further, cooling balloons of the prior art have been found to have an expanded conformation wherein the middle portion of the balloon has a larger circumference than the end portions.

Such an expanded conformation can result in distention of the atrium during surgery, producing undesirable damage to anatomical structures. Still even further, it has been found that currently employed heat transfer media are incapable of cooling the heart to optimally advantageous low temperatures. Greater cooling of the right atrium would reduce its electrical activity and help to reduce postoperative atrial fibrillation. Likewise, cooling of the sinoatrial node to lower temperatures than is possible with currently employed heat transfer media is highly advantageous in further reducing its electrical activity.

Options, alternatives and technically unique alternatives, readily have been used extensively in cardiac surgery, yet it is still the case that prior devices, products, or methods such as these available to medical practitioners have not adequately addressed the need for advanced methods and apparatus for minimizing the deficiencies in heat regulation and minimizing the potential for damage to cardiac anatomical structures as set forth above. The present invention embraces and finally addresses the clear need for advanced methods and apparatus for solving the long-standing needs in atrial cooling as set forth above. Those skilled in the art are well versed in the cross-functional approaches and advances ranging from the most to least invasive procedures, on- and off-pump methods and the like treatment schemes by which the instant teachings may be actualized and actuated.

Thus, as pioneers and innovators attempt to make methods and apparatus for cooling cannulae more effective, more universally used, and of higher quality, none has approached the desiderata outlined above in combination with simplicity and reliability of operation, until the teachings of the present invention. It is respectfully submitted that other references merely define the state of the art or show the type of systems that have been used to alternately address those issues ameliorated by the teachings of the present invention. Accordingly, further discussions of these references has been omitted at this time due to the fact that they are readily distinguishable from the instant teachings to one of skill in the art.

### OBJECTS AND SUMMARY OF THE INVENTION

An object of the present invention is to provide novel enhanced means for cooling select aspects of a patient's vasculature whereby cell, tissue and organs system is mitigated, extenuated or prevented.

A further object is to apply an improved cooling technique to procedures designated to address, ameliorate, extenuate, mitigate, or prevent tissue insult and injury within the context of therapies for HVD, CAD, CHF, and PVD or the like disease states.

Clearly, CPB is a complex procedure, and it is subject to limitations and disadvantages that can contribute significantly to patient mortality, patient morbidity, and healthcare costs. It is therefore highly desirable, and a major object of this invention, to provide improved CPB devices and methods that make possible less traumatic, safer, and more cost-effective procedures.

It is an object of the present invention to provide a cooling cannula system that facilitates “flipping”, or rotating the hear, so that the backside is exposed during surgery without a substantial risk of damaging structures within the heart during this maneuver. It is another object of the present invention to provide a cooling cannula system effective to produce a leak-free isolation of the venae cavae. It is still another object of the present invention to provide a cooling cannula system that does not produce substantial blockage of the coronary sinus during surgery.

It is yet still another object of the present invention to provide a cooling cannula system-that includes a cooling member having an expanded conformation wherein the middle portion has a circumference no greater than the end portions.

It is even yet still another object of the present invention to provide a cooling cannula system lacking a substantial risk of infringing and hence damaging the tricuspid valve.

It is a further object of the present invention to provide a cooling cannula system that can provide cardiac cooling to lower temperatures than is possible with currently employed heat transfer media.

It is even a further object of the present invention to provide a cooling cannula system embodying a solid-state thermoelectric cooling device utilizing the Peltier effect.

It is yet a further object of the present invention to provide a cooling cannula system that can provide sinoatrial node cooling to lower temperatures than is possible with currently employed heat transfer media.

It is yet still another further object of the present invention to provide a cooling cannula system that effectively reduces post-operative atrial fibrillation.

It is even still a further object of the present invention to provide a cooling cannula system incorporating a cooling member possessing circumferential bands for shape control.

It is even yet still a further object of the present invention to provide a cooling cannula system adapted for embodiments that are capable of direct insertion via the right atrium or peripheral insertion via a vein.

These and other objects are accomplished by the parts, constructions, arrangements, combinations and subcombinations comprising the present invention, the nature of which is set forth in the following general statement, and preferred embodiments of which—illustrative of the best modes in which applicant has contemplated applying the principles—are set forth in the following description and illustrated in the accompanying drawings, and are particularly and distinctly pointed out and set forth in the appended claims forming a part hereof.

The present invention is directed to improved cooling cannula systems and methods for their use in heart surgery handling to treat at least one of HVD, CAD, CHF, and PVD or the like disease states. The present invention may exist in numerous embodiments, including those that may be inserted peripherally thus avoiding the need for a major chest incision such a thoracotomy or median sternotomy.

By utilizing fluoroscopic or ultrasound imaging, the cannula may be precisely positioned such that upon inflation of inflatable members such as balloons, the flow of blood into the right atrium is fully blocked thereby achieving total CPB. The cooling cannula may use conventional heat transfer media cooled by refrigeration systems known in the art, or it may use thermoelectric cooling devices to accomplish the desired cooling.

By way of background, and in no way limiting the instant teachings, which cover numerous cooling means and modalities, Thermoelectric coolers are solid-state heat pumps that operate on the Peltier effect, the theory that there is a heating or cooling effect when electric current passes through two conductors. A voltage applied to the free ends of two dissimilar materials creates a temperature difference. With this temperature difference, Peltier cooling will cause heat to move from one end to the other. A typical thermoelectric cooler will consist of an array of p- and n-type semiconductor elements that act as the two dissimilar conductors. The array of elements is soldered between two ceramic plates, electrically in series and thermally in parallel. As a dc current passes through one or more pairs of elements from n- to p-, there is a decrease in temperature at the junction (“cold side”) resulting in the absorption of heat from the desired structure. The heat is carried through the cooler by electron transport and released on the opposite (“hot”) side as the electrons move from a high to low energy state. The heat pumping capacity of a cooler is proportional to the current and the number of pairs of n- and p-type elements (or couples).

In accordance with one embodiment of the invention, there is provided a cooling cannula comprising an insertion piece for insertion into the right atrium through the superior vena cava. The insertion piece has a plurality of apertures for drainage of the inferior vena cava at its distal end, and is joined at its proximal end to the distal end of a connection piece. The connection piece is fitted at its proximal end with a coupling to a suction device in a heart-lung machine. The apertures collectively comprise a cross-section sufficient to accommodate the blood being bypassed.

The above described, currently clinically utilized version of the instant teachings likewise feature a cannula which is preferably bent to form an angle between about a right angle and an obtuse angle of about 110° (an angle of inclination from linearity of about 70°). The insertion piece has a side opening positioned so as to drain the superior vena cava. In other embodiments the device can be constructed to enable insertion either through the inferior vena cava, or peripherally.

In embodiments designed to be inserted through the inferior vena cava, the plurality of apertures drains the superior vena cava, and the side opening drains the inferior vena cava. In embodiments designed to be inserted through the femoral vein, the plurality of apertures drains the superior vena cava, and the side opening drains the inferior vena cava. In embodiments designed to be inserted through the jugular vein, the plurality of apertures drains the inferior vena cava, and the side opening drains the superior vena cava.

The internal diameter of the insertion piece and the internal diameter of the connecting piece are proportional to the volume of the transported bloodstream. The invention provides an improved means for cooling luminal surfaces of the atrium involved in CPB procedures. In one embodiment, the means for cooling comprise a radially expandable cooling membrane which in one expanded predetermined configuration has substantial engagement with the thick tissue shelves of the right atrium, but does not have substantial contact with the thin tissue appendages, tricuspid valve, coronary sinus, and other interior surfaces of said right atrium. In this way, damage to the thin tissue appendages, tricuspid valve, coronary sinus, and other interior surfaces of the right atrium is avoided. Moreover, traction on the sinoatrial node is prevented by the cannula. Furthermore, the supraventricular excitor and conduction systems are protected, thus minimizing heart rhythm and conduction disturbances.

The invention provides cooling means for the thin tissue appendages, tricuspid valve, coronary sinus, and other interior surfaces of said right atrium. In this way, damage to the thin tissue appendages, tricuspid valve, coronary sinus, and other interior surfaces of the right atrium is avoided. The cooling means, in a preferred embodiment, comprise an inflatable membrane that has a separate inlet and outlet duct to allow a continual flow. By achieving a balance between inflow and outflow of coolant, the inflatable membrane can be kept inflated. The inventors believe, but are not certain, that a thin layer of liquid or blood between the membrane and the luminal structures of the right atrium is sufficient to enable sufficient heat transfer to account for the observed cooling.

The heat radiation from the cannula in the right atrium is also absorbed by the inflatable membrane. The inflatable membrane, which is made of a biocompatible polymer such as polyurethane, may be made more conductive by the incorporation of particles of a biocompatible metal, or particles of a biocompatible metal alloy comprising at least two elements selected from the group consisting of iron, cobalt, chromium, nickel, titanium, niobium, and molybdenum. Alternatively, the same metals or alloys can be formed into sheets to coat both the interior and exterior surfaces of said inflatable membrane.

Further objects and advantages of the invention will become apparent to those skilled in the art upon reading and understanding the following detailed description and the accompanying drawings. It should be understood that the drawings are not necessarily to scale, that they show only certain embodiments of the invention, that certain details not essential to an understanding of the invention may have been omitted, and that like numbers indicate like structures.

### BRIEF EXPLANATION OF THE DRAWINGS

Those having a modicum of skill in the treatment of HVD, CAD, CHF, PVD, and the like disease states will understand modifications of the exemplary structure offered herein merely for illustrative purposes, and realize that such illustrated embodiments enable, but in no way, limit the instant teachings.

FIG. 1 is a schematic cut-away view of a cooling cannula, according to the invention, inserted via an opening in the superior vena cava.

FIG. 2 is an enlarged, cut-away, elevational view of a cooling cannula system of the present invention.

FIG. 3 is an enlarged cross-sectional view through line 3-3 of FIG. 2.

FIG. 4 is an enlarged cross-sectional view through line a-a of FIG. 2.

FIG. 5 is an enlarged elevational view of a cooling cannula system of the present invention.

FIG. 6 is an elevational view of a bifurcated cooling cannula system of the present invention.

### DETAILED DESCRIPTION OF THE PREFERRED EMBODIMENTS

The following detailed description is provided for the purpose of describing and illustrating presently preferred embodiments of the invention only, and is not intended to exhaustively describe all possible embodiments in which the invention may be practiced. As discussed, unique aspects of CAD, HVD, CHF, PVD and the like disease states drive modifications of the described devices and methods such the artisans would be able to effect such changes, given the guidance of those descriptions read along with the claims appealed hereto.

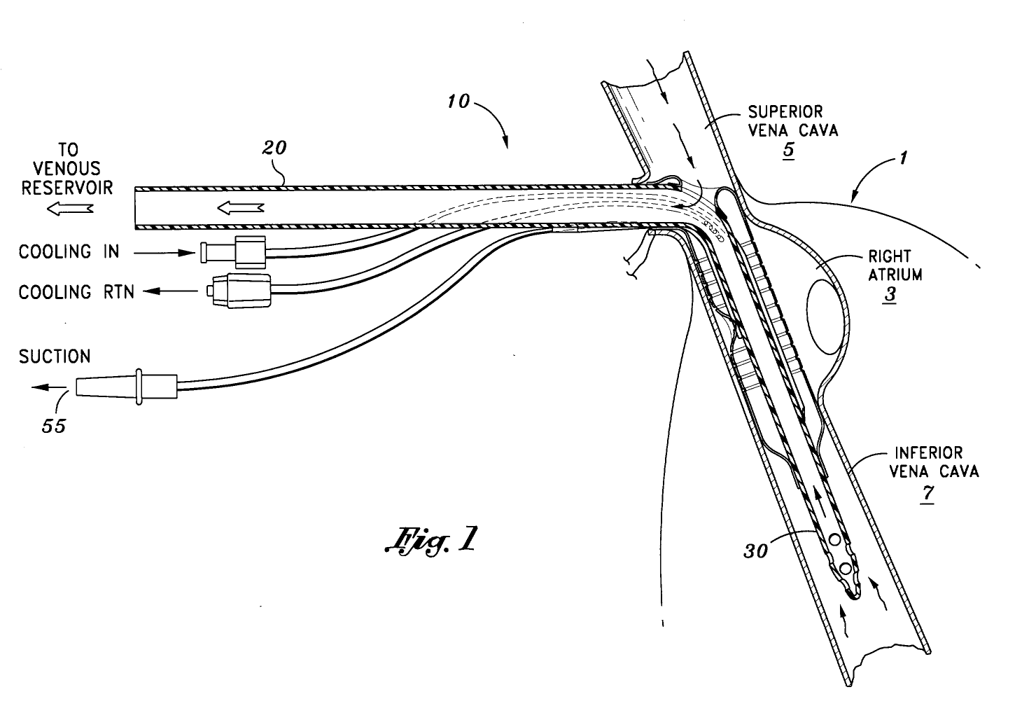

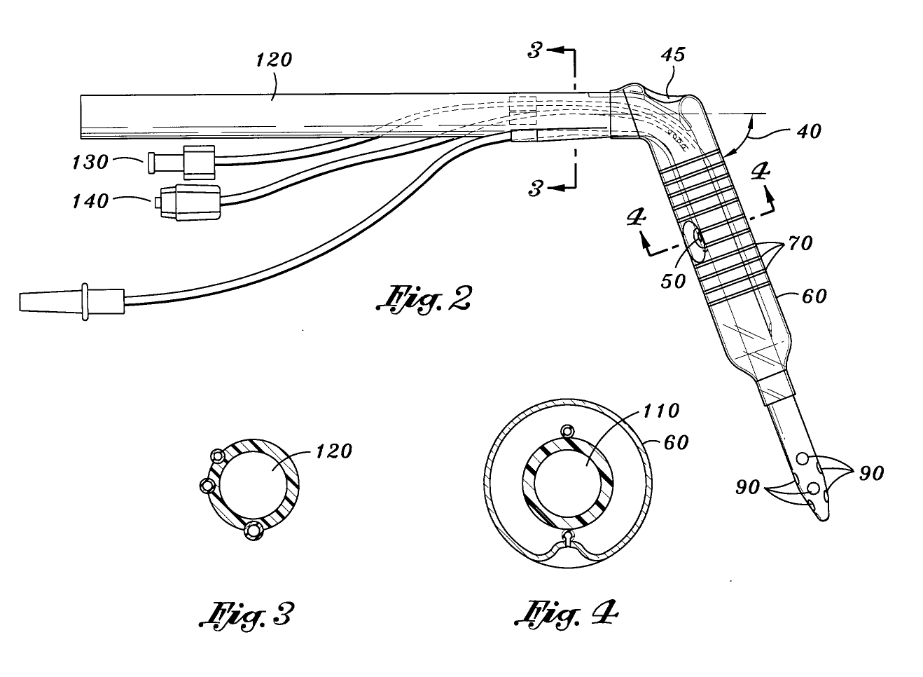

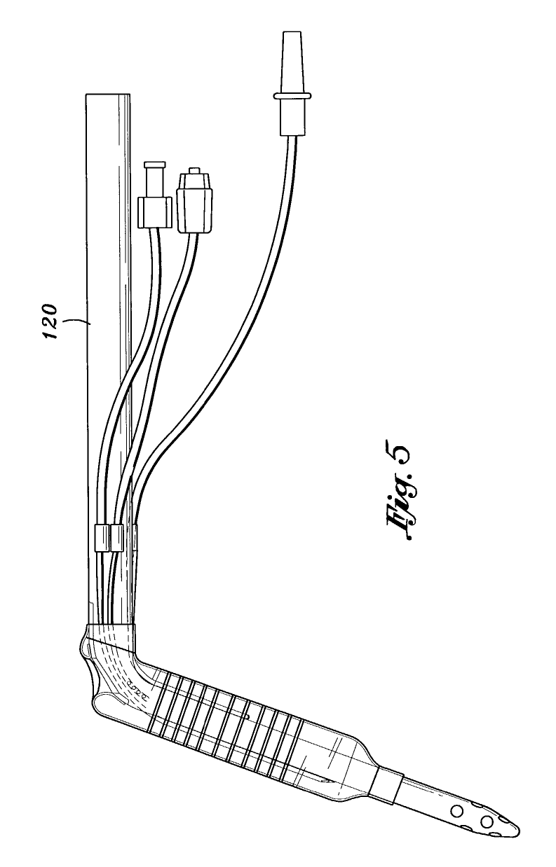

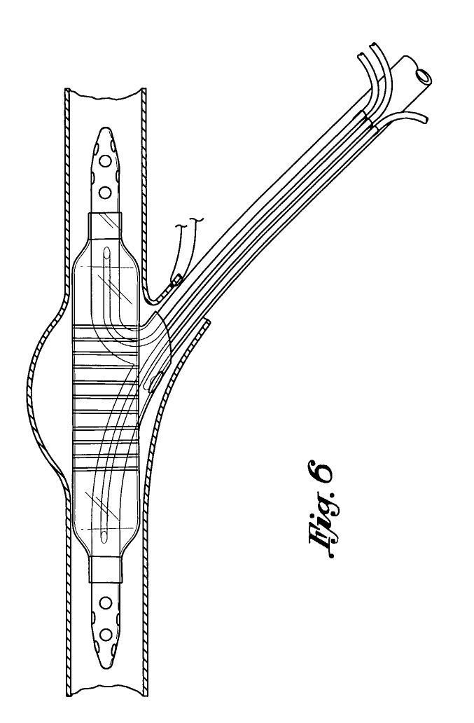

Referring to FIGS. 1, 2, 3, 4, and 5, a portion of a heart, generally indicated by 1, includes the right atrium 3, the superior vena cava 5, and the inferior vena cava 7. During an operation where heart function is disconnected, and the circulation is taken over by a heart-lung machine, blood is extracted at the point where the superior and inferior venae cavae join.

A cannula 10 having an outer diameter between about 20 Fr and about 55 Fr is used for this purpose, and may include fluid communication access adapted for venous blood pressure measurement of said patient. Cannula 10 consists of a connecting piece 20 and a thinner insertion piece 30. Connecting piece 20 and insertion piece 30 are joined to each other by an angle of inclination 40. Angle 40 can range between approximately 50° and approximately 90° depending on the physical properties of the material used to make cannula 10. When such material is flexible and can be bent without occluding the lumen of the cannula, an angle smaller than about 50° is permissible. Preferably, the angle is between about 60° and 80°, and even more preferably between about 65° and about 75°. An opening 45, which provides a connection with the superior vena cava, is positioned in this angle. The distal end of insertion piece 30 is provided with at least one hole 90. The cross sectional area of hole 90 should be sufficiently large to accommodate the flow volume in the cannula.

When in use, insertion piece 30 is inserted into superior vena cava 5, passed through right atrium 3, and extends into inferior vena cava 7. According to the invention, means for reinforcement such as metal spirals or other reinforcement means known in the art are positioned to prevent buckling of the material from which the connecting piece 20 and the insertion piece 30 are made. Venous drainage of inferior vena cava 7 passes through hole 90, and then through a lumen 110 in insertion piece 30, and then through a lumen 120 in connecting piece 20. Venous drainage of superior vena cava 5 passes through hole 45, and then through a lumen 125 in connecting piece 20. Connecting piece 20 is connected at its proximal end to a venous reservoir, not shown.

According to the invention, cannula 10 is provided with cooling means. These consist of an inlet for coolant 130 and an outlet for coolant 140, which are connected to a radially expandable membrane constructed of a biocompatible polymer such as polyurethane, silicone, latex, polyvinylchloride, polyolefin, low-density polyethylene, and polycarbonate. The coolant may be any suitable heat transfer fluid including water, aqueous saline solution, and calcium chloride solution and perfluorocarbon fluid. The membrane has at least one unexpanded predetermined configuration and at least one expanded predetermined configuration, which may comprise a substantially cylindrical form.

When expanded, one end of the substantially cylindrical form may comprise a larger circumference than other portions. The substantially cylindrical form may have a length between about 6 cm and about 14 cm, wherein the middle portion has a maximum diameter between about 15 mm and about 30 mm. Sections of the membrane may comprise at least two different thicknesses and may include a substantially cylindrical middle portion having at least one circumferential ring 70 formed on its inner surface by thickened sections of biocompatible polymer. The membrane is in the form of an inflatable balloon 60 in one embodiment of the invention. A passage 50 through balloon 60 is sealingly connected in fluid communication with right atrial suction tube 55. When suction is applied to tube 55, excess cardioplegic fluid used in the procedure is drained from right atrium 3 through passage 50 into tube 55, which is connected to a suction means such as a vacuum pump, not shown.

Balloon 60 may be wrapped around insertion piece 30 so that it is possible to insert it easily into superior vena cava 5. By applying coolant pressure in the balloon the balloon is inflated and substantially engages the thick tissue shelves of right atrium 3, superior vena cava 5 and inferior vena cava 7 in order to occlude and cool them. By contrast, there is little or no contact with the thin tissue appendages, tricuspid valve, coronary sinus, and other interior surfaces of right atrium 3.

The expandable membrane may be produced by fabricating a substantially cylindrical mandrel having a circumference substantially equal to the circumference of the substantially cylindrical middle portion of the expandable membrane in its expanded configuration, fabricating at least one circumferential groove in the mandrel in areas corresponding to the substantially cylindrical middle portion of the expandable membrane, immersing the mandrel in a liquid dispersion of a biocompatible polymer, removing the mandrel from the dispersion, curing the biocompatible polymer on the mandrel; and, removing the solidified membrane. Alternatively, the membrane can be made by blow molding.

To facilitate heat transfer, the polymer may incorporate dispersed particles of biocompatible metal such as iron, cobalt, chromium, nickel, titanium, niobium, silver, gold, platinum, aluminum, and molybdenum, or a biocompatible metal alloy comprising at least two elements selected from the group consisting of iron, cobalt, chromium, nickel, titanium, niobium, silver, gold, platinum, aluminum, and molybdenum. Alternatively, such metal and metal alloys can be combined with the polymer in the form of layers.

Likewise, alternative cooling means will be well known to artisans, including at least the following; chemical by means an some manner of endothermic reaction: the mixture of two chemicals that become cold when reacted (for example, an emergency cooling packs). A mechanical, or solvent/solute based reaction set, such as by using a pre-cooled gel that requires no circulating circuit (as in a pre-frozen gel packs).

One could likewise inject chemical means, precursor means, media or related forms of an appropriate composition of matter, compound, or material (for example DMSO/RIMSO50® brand as distributed by Edwards Lifesciences, Utah, USA) that has high freezing temperature. Those skilled also understand the electrical way, as in the Peltier example given, which only illustrates one of the many known techniques used recently. Such mechanisms could include a refrigerant—external compressor and evaporator and/or biochemical means from iontophoresis to some way to promote lower ATP usage, or evaporative, or Conductive mechanisms to cool the air external to appropriate anatomy via Vortex tube.

Another alternative approach whereby at least one of the above cooling means could be used to cool the significant anatomy internally (a device inserted into the RA), or externally is within the scope of the instant teachings. All above cooling means could be combined with venous drainage cannulae or as stand-alone devices, according to the present invention.

A method of use for providing drainage of venous blood using the invention comprises opening the vasculature, draining venous blood from at least one of the right atrium, superior vena cava and inferior vena cava; and, cooling involved luminal surfaces; whereby tissue insult and injury is partially mitigated. A radiopaque area (such as barium sulfate or stainless steel) may be situated near the free end of the tubular insertion section. This modification enables the surgeon to provide drainage of venous blood from a targeted location without opening the right atrium or the chest of a patient undergoing cardiac surgery. The method involves identifying a targeted location, inserting a guidewire in a jugular vein or femoral vein of a patient, observing the placement of the guidewire fluoroscopically while passing the guidewire through the vein until the distal end of the guidewire is situated in the targeted location; threading the proximal end of the guide wire through the free end of the cannula; passing the cannula along the guidewire while observing fluoroscopically until the radiopaque area lies in the targeted location; withdrawing the guidewire; draining venous blood and cooling involved luminal surfaces.

An article of manufacture, comprising packaging material and the novel enhanced cardiac surgical cooling system contained within the packaging material, wherein the novel enhanced cardiac surgical cooling system is effective for cardiac surgery in a patient afflicted with cardiac disease, and the packaging material includes a label that indicates that said device is effective for said cardiac surgery.

On this basis, the instant invention should be recognized as constituting progress in science and the useful arts, as solving the problems in cardiology enumerated above. In the foregoing description, certain terms have been used for brevity, clearness and understanding, but no unnecessary limitation are to be implied therefrom beyond the requirements of the prior art, because such words are used for descriptive purposes herein and are intended to be broadly construed.

Having described preferred embodiments of the invention with reference to the accompanying drawings, it is to be understood that the invention is not limited to those precise embodiments, and that the various changes and modifications may be effected therein by one skilled in the art without departing from the scope or spirit of the invention s defined in the appended claims. Various additions, deletions, alterations and modifications may be made to the above-described embodiments without departing from the intended spirit and scope of the invention. It is, for example, possible to use all sorts of other cooling means, such as tubing wrapped around the cannula, or other structures known in the art around a tube for efficient transfer of heat (cold) to the surroundings, wherein insulation measures ensure that the contents of the tube are not affected. Furthermore, warming can done instead of cooling. Thus, the scope of the invention should be determined by the appended claims and their legal equivalents, rather than by the examples given. All changes that come within the meaning and range of equivalency of the claims are to be embraced within their scope.

Definitions

Unless defined otherwise, all technical and scientific terms used herein have the same meaning as is commonly understood by one of skill in the art to which this invention belongs. All patents and publications referred to herein are incorporated in their entirety by reference, including expressly U.S. Pat. No. 5,562,606 and all of its foreign equivalents.

What is claimed is:

1. In a novel enhanced cardiac surgical cooling system, the improvements comprising, in combination:
a first means for draining venous blood from at least one of the right atrium, superior vena cava and inferior vena cava; and,
an improved means for cooling involved luminal surfaces, whereby tissue insult and injury is partially mitigated by engagement of said means for cooling with select aspects of involved atrial tissue to facilitate transfer of heat.

2. The novel enhanced cardiac surgical cooling system of claim 1, wherein:
said first means comprises at least a cooling system membrane having a cannula adapted for emplacement in fluid communication with said at least one of the right atrium, superior vena cava and inferior vena cava;
said at least a cooling system membrane includes at least one aperture effective for direct drainage of said at least one of the right atrium, superior vena cava and inferior vena cava; and,
said at least a cooling system membrane is in fluid communication with a suction means for applying reverse pressure.

3. The novel enhanced cardiac surgical cooling system of claim 2, said at least a cooling system membrane further comprising:
a radially expandable membrane having at least a first and a second configuration, wherein said membrane is adapted to facilitate transfer of heat when positioned in predetermined spatial orientation relative to said involved luminal surfaces.

4. The novel enhanced cardiac surgical cooling system according to claim 2, wherein said cannula has an outer diameter between about 20 Fr and about 55 Fr.

5. The novel enhanced cardiac surgical cooling system of claim 3, wherein said improved means for cooling further comprises:
said expandable membrane adapted to receive a heat transfer means;
said membrane having at least one unexpanded predetermined configuration and at least one expanded predetermined configuration;
said membrane in said at least one expanded predetermined configuration comprising a substantially cylindrical form; wherein:
at least one end of said membrane in said at least one expanded predetermined configuration comprises a larger circumference than other portions of said substantially cylindrical form;
so that in operation during cardiac surgery, said membrane in said at least one expanded predetermined configuration has substantial engagement with the thick tissue shelves of said at least one of the right atrium, superior vena cava and inferior vena cava; wherein said substantial engagement is adapted to occlude and cool said at least one of the right atrium, superior vena cava and inferior vena cava; and, said membrane in said at least one expanded predetermined configuration has insubstantial contact with the thin tissue appendages, tricuspid valve, coronary sinus, and other interior surfaces of said right atrium.

6. The novel enhanced cardiac surgical cooling system according to claim 3, wherein said expandable membrane is constructed of a biocompatible polymer.

7. The novel enhanced cardiac surgical cooling system of claim 4, wherein said membrane in said at least one expanded predetermined configuration has a length between about 6 cm and about 14 cm, and said middle portion has a maximum diameter between about 15 mm and about 30 mm.

8. The novel enhanced cardiac surgical cooling system according to claim 5, wherein said expandable membrane in said at least one expanded predetermined configuration includes a substantially cylindrical middle portion having sections of said biocompatible polymer wherein said sections comprise at least two different thicknesses.

9. The novel enhanced cardiac surgical cooling system according to claim 5, wherein said expandable membrane in said at least one expanded predetermined configuration includes a substantially cylindrical middle portion having at least one circumferential ring formed on its inner surface by thickened sections of said biocompatible polymer.

10. The novel enhanced cardiac surgical cooling system according to claim 1, further including fluid communication access adapted for venous blood pressure measurement of said patient.

11. The novel enhanced cardiac surgical cooling system according to claim 3, wherein said expandable membrane has at least one connection for a heat transfer fluid.

12. The novel enhanced cardiac surgical cooling system according to claim 11, wherein said expandable membrane has an inlet connection for supplying said heat transfer fluid to said expandable membrane, and an outlet connection for draining said heat transfer fluid from said expandable membrane.

13. The novel enhanced cardiac surgical cooling system according to claim 11, wherein said heat transfer fluid is selected from the group consisting of water, aqueous saline solution, calcium chloride solution and perfluorocarbon fluid.

14. The novel enhanced cardiac surgical cooling system according to claim 6, wherein said polymer is selected from the group consisting of polyurethane, silicone, latex, polyvinylchloride, polyolefin, low density polyethylene, and polycarbonate.

15. A method for making the expandable membrane as defined in claim 9, said method comprising the steps of:
fabricating a substantially cylindrical mandrel having a circumference substantially equal to said circumference of said substantially cylindrical middle portion of said expandable membrane in said at least one expanded predetermined configuration;
further fabricating at least one circumferential groove in said mandrel in areas corresponding to said substantially cylindrical middle portion of said expandable membrane;
immersing said mandrel in a liquid dispersion of said biocompatible polymer;
removing said mandrel from said liquid dispersion of said biocompatible polymer;
curing said biocompatible polymer on said mandrel; and,
removing said solidified membrane;
whereby said expandable membrane having a substantially cylindrical middle portion having at least one circumferential ring formed by at least one thickened section of said biocompatible polymer is obtained.

16. A method for making the expandable membrane as defined in claim 9, said method comprising the steps of:
fabricating a mold adapted for blow molding said substantially cylindrical middle portion of said expandable membrane in said at least one expanded predetermined configuration; and,
blow molding said expandable membrane.

17. Product, obtained by the process of claim 15.

18. Product, obtained by the process of claim 16.

19. The novel enhanced cardiac surgical cooling system according to claim 6, wherein said polymer incorporates dispersed metal particles.

20. The novel enhanced cardiac surgical cooling system according to claim 19, wherein said metal particles are selected from the group consisting of particles of a biocompatible metal selected from the group consisting of iron, cobalt, chromium, nickel, titanium, niobium, silver, gold, platinum, aluminum, and molybdenum, and particles of a biocompatible metal alloy comprising at least two elements selected from the group consisting of iron, cobalt, chromium, nickel, titanium, niobium, silver, gold, platinum, aluminum, and molybdenum.

21. The novel enhanced cardiac surgical cooling system according to claim 6, wherein said polymer is selected from the group consisting of polymers that have a metal layer formed on one surface; and, polymers that have a metal layer formed between an interior surface and an exterior surface.

22. The novel enhanced cardiac surgical cooling system according to claim 21, wherein said polymer has a metal layer formed on all of its surfaces.

23. The novel enhanced cardiac surgical cooling system according to claim 21, wherein said metal layer is selected from the group consisting of a layer of a biocompatible metal and a layer of a biocompatible metal alloy comprising at least two elements selected from the group consisting of iron, cobalt, chromium, nickel, titanium, niobium, and molybdenum.

24. A method for providing drainage of venous blood using the novel enhanced cardiac surgical cooling system according to claim 1 for a patient undergoing cardiac surgery, said method comprising the steps of:
opening the vasculature;
draining venous blood from at least one of the right atrium, superior vena cava and inferior vena cava; and,
cooling involved luminal surfaces;
whereby tissue insult and injury is partially mitigated by engagement of said means for cooling with select aspects of involved atrial tissue to facilitate transfer of heat.

25. The novel enhanced cardiac surgical cooling system of claim 2, further comprising a radiopaque area situated near the free end of said tubular insertion section.

26. The novel enhanced cardiac surgical cooling system of claim 25, wherein said radiopaque area is made radiopaque with a radiopaque with a substance selected from the group consisting of barium sulfate and stainless steel.

27. A method for providing drainage of venous blood from a targeted location in without opening the right atrium or the chest of a patient undergoing cardiac surgery, said method comprising the steps of:
identifying said targeted location;
inserting a guidewire in a vein selected from the group consisting of the jugular vein and the femoral vein of said patient;
observing the placement of said guidewire fluoroscopically while passing said guidewire through said vein until the distal end of said guidewire is situated in said targeted location;
threading the proximal end of said guide wire through said free end of said cannula of said novel enhanced cardiac surgical cooling system according to claim 25;
passing said novel enhanced cardiac surgical cooling system along said guidewire while observing the placement of said novel enhanced cardiac surgical cooling system fluoroscopically until said radiopaque area lies in said targeted location;
withdrawing said guidewire;
draining venous blood from said at least one of the right atrium, superior vena cava and inferior vena cava; and,
cooling involved luminal surfaces;
whereby tissue insult and injury is partially mitigated by engagement of the means for cooling with select aspects of involved atrial tissue to facilitate transfer of heat.

28. An article of manufacture, comprising packaging material and the novel enhanced cardiac surgical cooling system according to claim 1 contained within the packaging material, wherein said novel enhanced cardiac surgical cooling system is effective for cardiac surgery in a patient afflicted with cardiac disease, and the packaging material includes a label that indicates that said device is effective for said cardiac surgery.

29. A novel enhanced intraluminal cooling system, comprising, in combination:
cannulae means for draining venous blood;
a communication means for facilitating transfer of at least a desired cooling medium; and,
actuating means permitting users to effect selective endothermic transfers.

30. The novel enhanced intraluminal cooling system of claim 29, said cannulae means further comprising at least a flexible alinear tubular assembly further defined by a plurality of transition zones.

31. The novel enhanced intraluminal cooling system of claim 30, wherein said communication means further includes at least a cooling system membrane in fluid communication with a suction means for applying reverse pressure.

32. The novel enhanced intraluminal cooling system of claim 31, said at least a cooling system membrane further comprising:
a radially expandable membrane having at least a first and a second configuration, wherein said membrane is adapted to facilitate transfer of heat when positioned in predetermined spatial orientation relative to said involved luminal surfaces, and any rotational forces created by expanding the membrane as it is expanded counteract each other, whereby rotational movement of the membrane beyond pre-defined limits is avoided.

33. The novel enhanced intraluminal cooling system of claim 32, wherein said cannulae means has an outer diameter ranging from at least about 18 Fr to at least about 60 Fr.

34. The novel enhanced intraluminal cooling system of claim 33, wherein said membrane in said at least one expanded predetermined configuration has substantial engagement with the thick tissue shelves of said at least one of the right atrium, superior vena cava and inferior vena cava; wherein said substantial engagement is adapted to occlude and cool said at least one of the right atrium, superior vena cava and inferior vena cava; and, said membrane in said at least one expanded predetermined configuration has insubstantial contact with the thin tissue appendages, tricuspid valve, coronary sinus, and other interior surfaces of said right atrium.

35. The novel enhanced intraluminal cooling system of claim 34, wherein said expandable membrane is constructed of a biocompatible polymer.

36. The novel enhanced intraluminal cooling system of claim 29, further comprising a radiopaque area situated near the free end of said cannulae means.

37. The novel enhanced intraluminal cooling system of claim 36, wherein said radiopaque area is made radiopaque with a radiopaque with a substance selected from the group consisting of barium sulfate and stainless steel.

38. The novel enhanced intraluminal cooling system according to claim 35, wherein said polymer incorporates dispersed metal particles.

39. The novel enhanced intraluminal cooling system according to claim 35, wherein said polymer is selected from the group consisting of polymers that have a metal layer formed on one surface; and, polymers that have a metal layer formed between an interior surface and an exterior surface.

40. The novel enhanced intraluminal cooling system according to claim 35, wherein said polymer is selected from the group consisting of polyurethane, silicone, latex, polyvinylchloride, polyolefin, low density polyethylene, and polycarbonate.

41. The novel enhanced intraluminal-cooling system according to claim 29, wherein said cooling medium is a heat transfer fluid.

42. The novel enhanced intraluminal cooling system according to claim 41, wherein said heat transfer fluid is selected from the group consisting of water, aqueous saline solution, calcium chloride solution and perfluorocarbon fluid.

43. The novel enhanced intraluminal-cooling system according to claim 29, further including fluid communication access adapted for venous blood pressure measurement.

44. An article of manufacture, comprising packaging material and the novel enhanced intraluminal cooling system according to claim 29 contained within the packaging material, wherein the packaging material includes a label that indicates that said intraluminal cooling system is effective for intraluminal cooling.

45. An enhanced cooling system for surgical procedures, which comprises, in combination:
A cannulae means for passing venous blood from a target area to a desired space; a first cooling means for lowering temperature of select tissue surfaces, and a second communication means for transferring at least the first cooling means from a predetermined location to a second location.

46. The enhanced cooling system is defined in claim 45, said cannulae means is angled for emplacement into the vasculature and has at least a plurality of apertures means and an angled tip at a first end.

47. The enhanced cooling system, as defined in claim 46, further comprising at least an inflatable member disposed substantially adjacent the cannulae means.

48. The enhanced cooling system, as defined in claim 46 wherein the first cooling means is at least one of mechanical, chemical, electrical, electromechanical, biochemical, and the like means for transferring heat.

49. The enhanced cooling system as defined in claim 48, wherein the system is used to treat at least one disease state selected from the group consisting of coronary artery disease, heart valve disease, congestive heart failure, peripheral vascular disease, and other sclerosis.

50. The cooling system of claim 49, said system capable of being emplaced endovascularly.
